(19) United States
(12) Patent Application Publication
(10) Pub. No.: US 2024/0260981 A1
(43) Pub. Date: Aug. 8, 2024

(54) INTRAVASCULAR LITHOTRIPSY CATHETER WITH MOVABLE EMITTERS

(71) Applicant: Shockwave Medical, Inc., Santa Clara, CA (US)

(72) Inventors: Rainier BETELIA, San Jose, CA (US); Todd JENKINS, San Jose, CA (US); Danielle ROND, Santa Clara, CA (US); Nancy P. NGUYEN, San Jose, CA (US); Jason B. FRANCO, Milpitas, CA (US)

(73) Assignee: Shockwave Medical, Inc., Santa Clara, CA (US)

(21) Appl. No.: 18/428,752

(22) Filed: Jan. 31, 2024

Related U.S. Application Data
(60) Provisional application No. 63/442,980, filed on Feb. 2, 2023.

(51) Int. Cl. A61B 17/22; A61B 17/00; A61B 90/00

(52) U.S. Cl. CPC A61B 17/22022; A61B 17/00234; A61B 90/08; A61B 2017/00305; A61B 2017/22001; A61B 2017/22025; A61B 2090/0811

(57) ABSTRACT

A catheter for treating an occlusion in a body lumen includes a movable emitter carrier having one or more shock wave emitters. The one or more shock wave emitters are movable in a longitudinal direction of the catheter along an inner shaft. In a first configuration of the catheter, the one or more shock wave emitters are retracted from a distal fluid-fillable enclosure. In a second configuration of the catheter, the one or more shock wave emitters are inserted in the distal fluid-fillable enclosure.

### PRIORITY

This application claims priority to U.S. Provisional Patent Application Ser. No. 63/442,980, filed Feb. 2, 2023, which is incorporated by reference in its entirety. This application is related to U.S. patent application Ser. No. ______ entitled “INTRAVASCULAR LITHOTRIPSY CATHETER WITH MOVABLE EMITTERS,” filed on Jan. 31, 2024, and having attorney docket No. 69026-20045.30, the entire disclosure of which is incorporated by reference.

### FIELD OF THE DISCLOSURE

The present disclosure relates generally to the field of medical devices and methods, and more specifically to shock wave catheter devices for treating calcified lesions in body lumens, such as calcified lesions and occlusions in vasculature and kidney stones in the urinary system.

### BACKGROUND

A wide variety of catheters have been developed for treating calcified lesions, such as calcified lesions in vasculature associated with arterial disease. For example, treatment systems for percutaneous coronary angioplasty or peripheral angioplasty use angioplasty balloons to dilate a calcified lesion and restore normal blood flow in a vessel. In these types of procedures, a catheter carrying a balloon is advanced into the vasculature along a guide wire until the balloon is aligned with calcified plaques. The balloon is then pressurized (normally to greater than ten atmospheres), causing the balloon to expand in a vessel to push calcified plaques back into the vessel wall and dilate occluded regions of vasculature. However, traditional angioplasty balloons are not always successful at dilating calcified lesions or vasculature, due to the stiffness and hardness of the calcified tissue and/or plaque.

More recently, catheters have been developed that include shock wave emitters (e.g., one or more electrode pairs) for generating shock waves inside an angioplasty balloon-this treatment is referred to as intravascular lithotripsy (“IVL”), a technology pioneered by Shockwave Medical, Inc., the applicant for the present application. Shock wave devices can be particularly effective for treating calcified lesions because the acoustic pressure from the shock waves can crack and disrupt lesions near the angioplasty balloon without harming the surrounding tissue. In these devices, the catheter is advanced over a guidewire through a patient's vasculature until it is positioned proximal to and/or aligned with the calcified lesion in a body lumen. The balloon is then inflated with conductive fluid (using a relatively low pressure of two to four atmospheres) so that the balloon expands to contact the lesion, and thereby apposition the shock wave emitters proximate to the lesion. The shock wave emitters can then be activated to produce acoustic shock waves that propagate through the walls of the angioplasty balloon and into the lesions. Once the lesions have been cracked by the acoustic shock waves, the balloon can be expanded further to increase the cross-sectional area of the lumen and improve blood flow through the vessel.

For electrohydraulic generation of acoustic shock waves, a conductive solution (e.g., saline) may be contained within an enclosure that surrounds electrodes or may be flushed through a lumen to the electrodes. The calcified plaque modification is achieved by creating acoustic shock waves within the catheter by an electrical discharge across the electrodes. The energy released by the electrical discharge enters the surrounding fluid faster than the speed of sound, generating an acoustic shock wave. The energy from the electrical discharge may also create a rapidly expanding and collapsing vapor bubble that results in an additional shock wave. These shock waves propagate radially outward and modify calcified plaque within the blood vessels. For laser generation of acoustic shock waves, a laser pulse is transmitted into and absorbed by a fluid within the catheter. This absorption process rapidly heats and vaporizes the fluid, thereby generating the rapidly expanding and collapsing vapor bubble and acoustic shock waves that propagate outward and modify the calcified plaque. The acoustic shock wave intensity is higher if a fluid is chosen that exhibits strong absorption at the laser wavelength that is employed. These examples of IVL devices are not intended to be a comprehensive list of potential energy sources to create IVL shock waves.

Traditional treatments for calcified lesions in patient vasculature often include mechanical alterations to the blood vessels, particularly atherectomy surgical procedures, which carry relatively greater risks of embolization (dislodgement of debris), abrupt closure (vessel collapse), dissection (tearing of vessel tissue), and/or perforation (puncturing of vessel tissue). The use of IVL in lieu of or in combination with traditional treatments for calcified lesions can greatly reduce the risk of embolization and perforation while concurrently leading to improved therapeutic outcomes in removing the calcified lesions.

Building on these advances, currently available shock wave catheters are limited by the length of the balloon and the position of emitters within that balloon. In patients where lesions are longer than the standard length of the balloon, a physician may elect to proceed with repeated cycles of inflation, emitting shock waves, deflation, and readjustment of balloon position in order to deliver shock wave treatment to the complete length of a given lesion. When used over repeated cycles, there is concern about the structural integrity of the balloon, and whether the balloon can retain its deflated profile for crossing a lesion. Further, any procedure or technique that adds time to a patient being on the table longer than needed is not well received in clinical cases because it can cause procedural complications.

Thus, longer balloons are desired by physicians to treat longer lesions to reduce procedure time and costs. Simply using a longer balloon may not suffice, as the distance from the emitters to relatively distant parts of a longer balloon may lead to insufficient shock wave strength at the extremities of the balloon. Further, it may not be practical or possible to simply increase the number of shock wave emitters as adding emitters tends to increase a catheter's profile and complicate its design. Such a catheter would also require a substantially higher energy output from a power supply. A high number of emitters can also reduce the flexibility and trackability of the balloon region of the catheter, limiting the treatment sites which can be reached.

Accordingly, there is an unmet need for catheter and emitter assembly designs capable of delivering shock waves for relatively long lesions within a patient, and accordingly minimizing the time a patient is in surgery receiving treatments.

### BRIEF SUMMARY

The above objects are realized in a catheter that includes a set of movable emitters that can be repositioned while deployed within a patient. More specifically, the innovation of present disclosure allows the end user (e.g., a physician) to physically move the emitters of an IVL device along the catheter inside relatively long balloons and position those emitters in various places alongside a long, calcified lesion as desired in order to disrupt calcium with shock wave technology.

Various examples can allow the user to keep very long balloons (e.g., balloons having working lengths 100-300 mm) inflated and in position in long lesions and instead move any number of emitters to desired location along the calcified lesion length inside the balloon that is inflated. This process can also be accomplished with the balloon slightly inflated, or semi-inflated depending on the calcium structure of the artery and there is no need to reposition the balloon at all.

In one or more embodiments, a catheter for treating an occlusion in a body lumen includes an IVL balloon with its emitters mounted on a movable tube.

In one or more examples, a catheter is configured for in situ customizable placement of emitters along a balloon length so that shockwave therapy can be applied optimally with minimal repositioning of the balloon required by the physician. Various embodiments of a catheter allow for efficient application of sonic energy to calcified regions of a vessel and avoids indiscriminately applying shock wave therapy to non-calcified regions of the vessel. Various embodiments also allow longer IVL balloons to be developed with relatively lower power requirements. Various embodiments of a catheter may improve flexibility, trackability, and/or deliverability of the catheter overall.

Various examples described herein allow the end user to physically move the IVL emitters inside very long balloons and position them in various places a long-calcified lesion as desired in order to disrupt calcium. According to an aspect of the disclosure, a catheter for treating an occlusion in a body lumen includes: an outer elongate member including a fluid lumen; a flexible enclosure secured to a distal region of the outer elongate member, the flexible enclosure being fillable with a conductive fluid via the fluid lumen; an inner elongate member, positioned within the outer elongate member and extending through the flexible enclosure to a distal region of the flexible enclosure; and a movable emitter member having an emitter assembly mounted thereon and connected to a power source, the movable emitter member located between the inner elongate member and the outer elongate member and movable in a longitudinal direction between the inner elongate member and the outer elongate member.

In a first configuration of the movable emitter member, the emitter assembly may be at a first location, and, in a second configuration of the movable emitter member, the emitter assembly may be at a second location more distal of the first location.

The emitter assembly may be located proximal of the flexible enclosure in the first configuration and within the flexible enclosure in the second configuration.

The flexible enclosure may have a working length l, the emitter assembly may include a plurality of shock wave emitters, and a distance between the most proximal shock wave emitter and the most distal shock wave emitter may be less than or equal to 0.5 l.

The catheter may further include a proximal hub. The outer elongate member, the inner elongate member, and the movable emitter member may extend to the proximal hub, and translation of the movable emitter member along the inner elongate member at the proximal hub may move the emitter assembly.

Translation of the movable member by a distance d at the proximal hub may move the emitter assembly by the distance d in the longitudinal direction.

The proximal hub may include a distal end opening fluidically sealed to a proximal end of the outer elongate member.

The inner elongate member of the catheter may include indicia that correlate to the longitudinal position of the emitter assembly within the flexible enclosure.

The proximal hub may include a distal diaphragm seal and a proximal diaphragm seal.

The inner elongate member and movable emitter member may each extend through the distal diaphragm seal. The inner elongate member may extend through the proximal diaphragm seal.

The proximal hub may include a position stabilizer with a first anchor at a first location of the hub and a second anchor at a second location of the hub more proximal of the first location and the movable emitter member includes a proximal end translatable between the first anchor and the second anchor.

At least one of the inner elongate member and the movable emitter member may include a polytetrafluoroethylene.

The inner elongate member may have an outer diameter d1 and the movable emitter member may include a lumen having a diameter d2 that is at least 0.002 inch greater than d1.

The emitter assembly may include an emitter centering member including: a proximal band; a distal band; and a plurality of flexible beams connecting the proximal ring to the distal ring, each strut having a central region that extends radially outward.

The flexible enclosure may include an inflated state and a deflated state, and, when the flexible enclosure is in an inflated state, the central region may extend radially farther outward than when the flexible enclosure is in a deflated state.

The emitter assembly may include one or more shock wave emitters and the central regions of the plurality of beams may space the emitters from a wall of the flexible enclosure.

The emitter centering member may include one or more of a polymer and a metal.

The flexible enclosure may include an angioplasty balloon having a working length l of at least 50 mm.

According to an aspect of the disclosure, a method of performing intravascular lithotripsy includes introducing a catheter into a body lumen, the catheter including: an elongate member extending from a catheter proximal end to a catheter distal end; a movable emitter member movable along the elongate member and comprising a shock wave emitter assembly; and an inflatable flexible enclosure secured to a distal region of the elongate member. The method further includes: advancing the catheter through the body lumen until the flexible enclosure is adjacent an occlusion of the body lumen; inflating the flexible enclosure with a fluid via a fluid lumen of the catheter; moving the emitter member along the elongate member; and supplying power to the shock wave emitter assembly to generate one or more shock waves.

Moving the emitter member may include moving the emitter member such that the shock wave emitter assembly moves between a first location proximal of the flexible enclosure and a second location within the flexible enclosure.

Moving the emitter member may include moving the emitter member such that the shock wave emitter assembly moves between a first location within the flexible enclosure and a second location within the flexible enclosure.

The method may further include imaging the body lumen with at least one of x-ray fluoroscopy, optical coherence tomography, and intravascular ultrasound.

Moving the emitter member may include moving a proximal end of the emitter member.

The method may further include correlating movement of the emitter assembly to movement of the emitter member at its proximal end by using indicia located along a proximal region of the elongate member.

According to an aspect of the disclosure, an emitter centering member for an IVL catheter includes: a proximal band; a distal band longitudinally spaced from the proximal band; and a plurality of flexible beams connecting the proximal ring to the distal ring, wherein each flexible beam includes a center region that compressibly extends laterally outward.

According to an aspect of the disclosure, a handle for an IVL device includes: a proximal end; an elongate member extending from the proximal end; a movable arm movably located on the elongate member and including a proximal diaphragm seal and a power port; a distal arm located distal of the movable arm and including a distal diaphragm seal and a fluid port; and a stabilizing bar that extends from the proximal end to the distal arm.

The movable arm may include a second fluid port.

The movable arm may include a third fluid port.

According to an aspect of the disclosure, a catheter for treating an occlusion in a body lumen includes: an elongate member including a fluid lumen; a flexible enclosure secured to a distal region of the outer elongate member, the flexible enclosure being fillable with a conductive fluid via the fluid lumen and having a working length l; and a movable emitter member having an emitter assembly mounted thereon and connected to a power source, the emitter assembly comprising a plurality of emitters, wherein a distance from a most proximal emitter to a most distal emitter is less than or equal to l/2 and the movable emitter member is longitudinally translatable within the flexible enclosure.

### DESCRIPTION OF THE FIGURES

Illustrative aspects of the present disclosure are described in detail below with reference to the following drawing figures. It is intended that the embodiments and figures disclosed herein are to be considered illustrative rather than restrictive.

FIG. 1A illustrates an exemplary catheter having a movable array of emitters inside an angioplasty balloon at a proximal position, according to aspects of the present disclosure.

FIG. 1B illustrates an exemplary catheter having a movable array of emitters inside an angioplasty balloon at a distal position, according to aspects of the present disclosure.

FIG. 1C illustrates a cross-sectional view of the exemplary catheter and one of the movable emitters inside an angioplasty balloon as shown in FIG. 1B.

FIG. 1D illustrates an exemplary wiring configuration for the exemplary catheter and movable emitters inside an angioplasty balloon, according to aspects of the present disclosure.

FIG. 1E illustrates an exemplary catheter having a movable array of emitters inside a lumen directly proximate to an angioplasty balloon, in a pair of configurations with the balloon inflated and deflated, according to aspects of the present disclosure.

FIG. 1F illustrates an exemplary catheter having a movable array of emitters inside a tapered angioplasty balloon at a distal position, according to aspects of the present disclosure.

FIG. 1G illustrates an exemplary catheter having a movable array of emitters inside an angioplasty balloon, configured for rapid exchange delivery, according to aspects of the present disclosure.

FIG. 1H illustrates an exemplary catheter having a movable array of emitters inside an angioplasty balloon, configured for rapid exchange delivery, according to aspects of the present disclosure.

FIG. 1I illustrates a cross-sectional view of an exemplary catheter having a movable array of emitters, according to aspects of the present disclosure.

FIG. 2A illustrates an exemplary catheter within a vascular structure, the catheter having a movable array of emitters inside an angioplasty balloon at a distal position, according to aspects of the present disclosure.

FIG. 2B illustrates an exemplary catheter within a vascular structure, the catheter having a movable array of emitters inside an angioplasty balloon at a central position, according to aspects of the present disclosure.

FIG. 2C illustrates an exemplary catheter within a vascular structure, the catheter having a movable array of emitters inside an angioplasty balloon at a proximal position, according to aspects of the present disclosure.

FIG. 3 is a flowchart describing a sequence of shock-wave treatments of lesions using a movable array of emitters within an angioplasty balloon, according to aspects of the present disclosure.

FIG. 4A illustrates an exemplary proximal handle for a catheter having a movable emitter assembly, according to aspects of the present disclosure.

FIG. 4B illustrates details for the proximal section the stabilizer structure shown in

FIG. 4A, according to aspects of the present disclosure.

FIG. 5A illustrates an exemplary proximal section of a movable emitter carrier, according to aspects of the present disclosure.

FIG. 5B illustrates an exemplary distal section of a movable emitter carrier, according to aspects of the present disclosure.

FIG. 6A illustrates an exemplary catheter having a movable emitter assembly with an emitter centering feature in a radially collapsed configuration, according to aspects of the present disclosure.

FIG. 6B illustrates the exemplary catheter of FIG. 6B with the emitter centering feature in a radially expanded configuration, according to aspects of the present disclosure.

FIG. 6C illustrates an exemplary catheter having a movable emitter assembly with an emitter centering feature, according to aspects of the present disclosure.

FIG. 6D illustrates an exemplary emitter centering structure in a radially collapsed configuration, according to aspects of the present disclosure.

FIG. 6E illustrates an exemplary emitter centering structure in a radially expanded configuration, according to aspects of the present disclosure.

FIG. 7 illustrates an exemplary catheter having an emitter centering feature, according to aspects of the present disclosure.

### DETAILED DESCRIPTION

The following description is presented to enable a person of ordinary skill in the art to make and use the various embodiments and aspects thereof disclosed herein. Descriptions of specific devices, assemblies, techniques, and applications are provided only as examples. Various modifications to the examples described herein will be readily apparent to those of ordinary skill in the art, and the general principles described herein may be applied to other examples and applications without departing from the spirit and scope of the various embodiments and aspects thereof. Thus, the various embodiments and aspects thereof are not intended to be limited to the examples described herein and shown but are to be accorded the scope consistent with the claims.

Efforts have been made to improve the design of electrode assemblies included in shock wave and directed cavitation catheters. For instance, low-profile electrode assemblies have been developed that reduce the crossing profile of a catheter and allow the catheter to more easily navigate calcified vessels to deliver shock waves in more severely occluded regions of vasculature. Examples of low-profile electrode designs can be found in U.S. Pat. Nos. 8,888,788, 9,433,428, and 10,709,462, and in U.S. Publication No. 2021/0085383, all of which are incorporated herein by reference. An example of a low-profile catheter with electrodes configured to be inserted after balloon expansion can be found in U.S. Pat. No. 10,357,264, which is incorporated herein by reference. Other catheter designs have improved the delivery of shock waves, for instance, by specific electrode construction and configuration thereby directing shock waves in a forward direction to break up tighter and harder-to-cross occlusions in vasculature. Examples of forward-firing catheter designs can be found in U.S. Pat. Nos. 10,966,737, 11,478,261, and 11,596,423 and U.S. Publication Nos. 2023/0107690 and 2023/0165598, all of which are incorporated herein by reference. U.S. Pat. No. 11,779,363, which is incorporated herein by reference, describes spacing of shock wave emitters to promote constructive interference of acoustic waves.

As used herein, the term “electrode” refers to an electrically conducting element (typically made of a metal or alloy) that receives electrical current and subsequently releases the electrical current to another electrically conducting element. Accordingly, as used herein, an “electrode pair” refers to two electrodes that are positioned adjacent to each other such that an electrical current provided to one electrode will transmit across the gap (also referred to as a “spark gap”) between the two electrodes (e.g., between a first electrode and a second electrode, or vice versa, optionally with an insulation separating the two electrodes, and with the electricity passing through a conductive fluid or gas therebetween). Further, as used herein, an “emitter” refers to a structure that has one or more electrode pairs. Emitters can be singular, paired, or otherwise arranged together to be electrically connected as an emitter assembly. Shock waves can be generated at each electrode pair of an emitter. In some contexts, one or more electrode pairs, which can be positioned across one or more emitters, can also be referred to as an “electrode assembly.”

In contrast to current balloons of similar length used clinically, these balloons as used for IVL applications may need to be formed of materials that have a higher tensile strength or more elasticity.

Typically, long calcified lesions in arteries and other vasculature presents challenges for many interventional devices and users. A user (e.g., a physician) will assess the length and diameter of calcified lesions, then determine the diameter and length of the balloon catheter that they want to use during the procedure. To treat relatively long calcified lesions, a user may prefer to use a single long balloon to treat such lesions, selecting balloons having a length of from about one hundred to three hundred millimeters (100 mm-300 mm) and having a diameter of from two to thirty millimeters when inflated.

In the context of current commercial IVL devices, the use of longer balloons presents a different catheter profile and thus different requirements to ensure adequate therapeutic function. In considering modifications to achieve therapeutic performance in a relatively long balloon, it is necessary to be mindful that there is a direct correlation between the number of emitters (given a fixed source of energy) and how effective IVL therapy is. In other words, as the number emitters increase to accommodate relatively long balloons for long lesions, the energy available to satisfy the increased number of emitters diminishes, and IVL efficacy may diminish as a result. This issue can be addressed in part by adding more channels to the IVL generator and system, although this solution results in a catheter that has more circuitry and wires, and thus a larger profile that may have more difficulty reaching smaller peripheral vasculature or crossing heavily occluded lesions. Current commercial IVL devices have two to five emitters within a balloon, where the emitters are adhesively bonded—and thus static in location—on the catheter shaft. Further, these devices have balloons with diameters from about twelve millimeters to sixty millimeters (12 mm-60 mm), where the working length of the balloon along the catheter is from one hundred ten centimeters to one hundred thirty-eight millimeters (110 mm-138 mm). This allows for a physician to go through a cycle of positioning, inflation, shock wave therapy, and deflation, moving the balloon over a catheter guidewire along the length of the lesion to treat the full length of the lesion. The emitters on these devices can pulse one at a time, two at a time, all at the same time, and/or pulse in an alternating or sequential pattern along the length of the catheter and balloon.

Described herein are catheters, balloons, and IVL circuits incorporating design elements that allow for treatment of relatively long calcified lesions, using an enclosure having an array of movable emitters. In such implementations, it is generally not necessary to go through an inflation and deflation cycle that requires repositioning the balloon for shock wave treatment as described above. Rather, the single balloon allows for movement of the emitters within the balloon to deliver shock wave treatment to target regions of a given long lesion. This can be accomplished with the long balloon slightly inflated or semi-inflated, depending on the calcium stricture within the artery or other vessel.

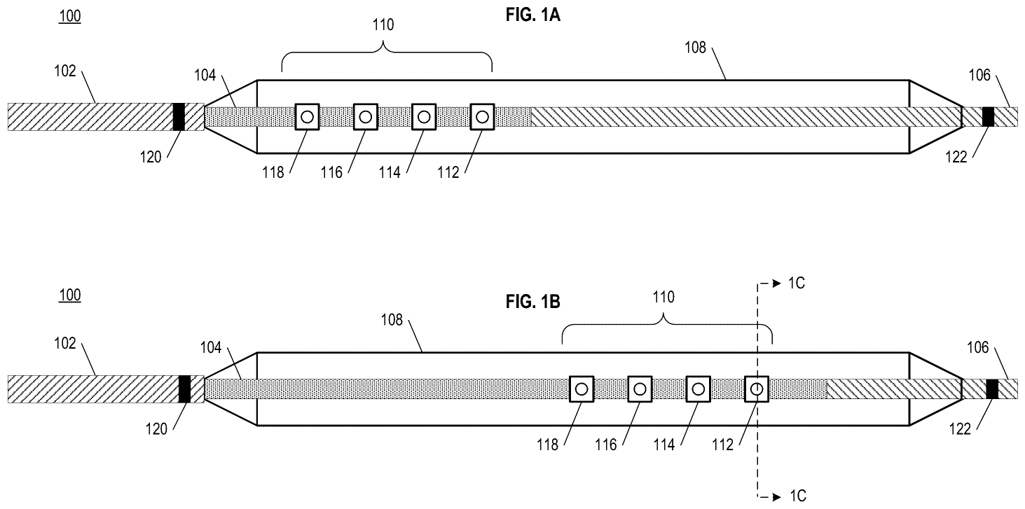

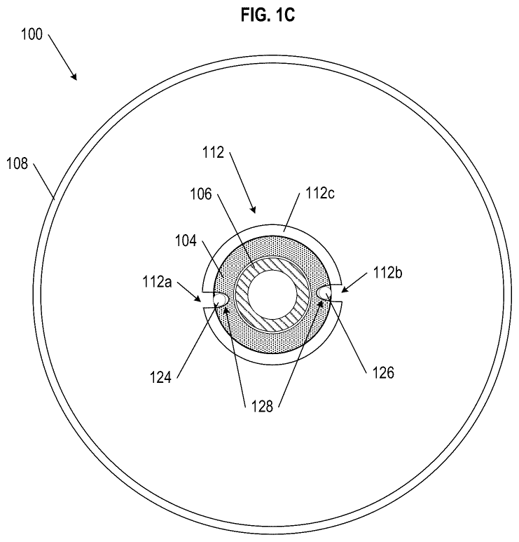

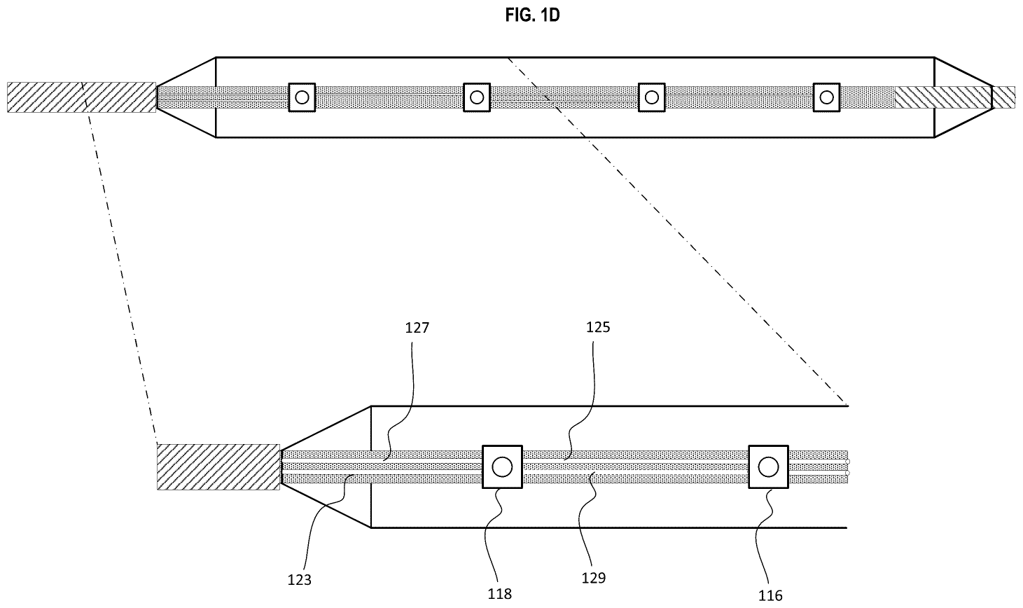

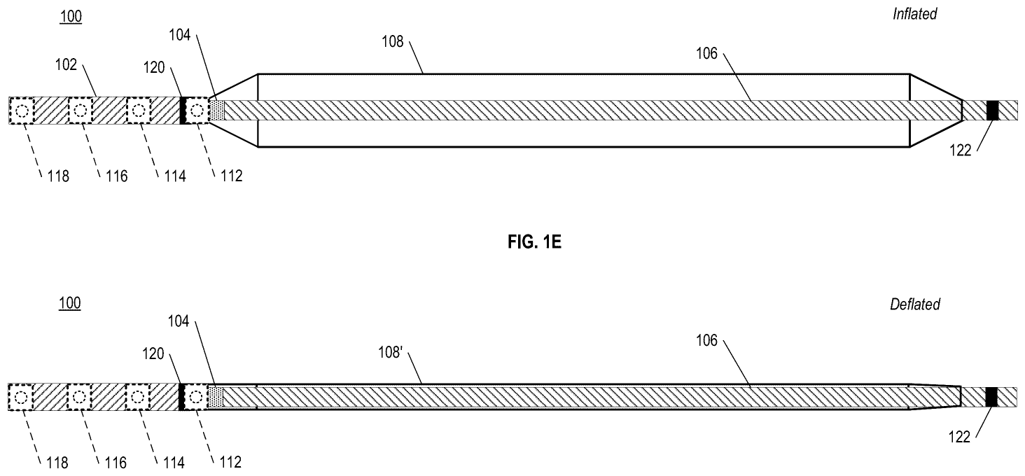

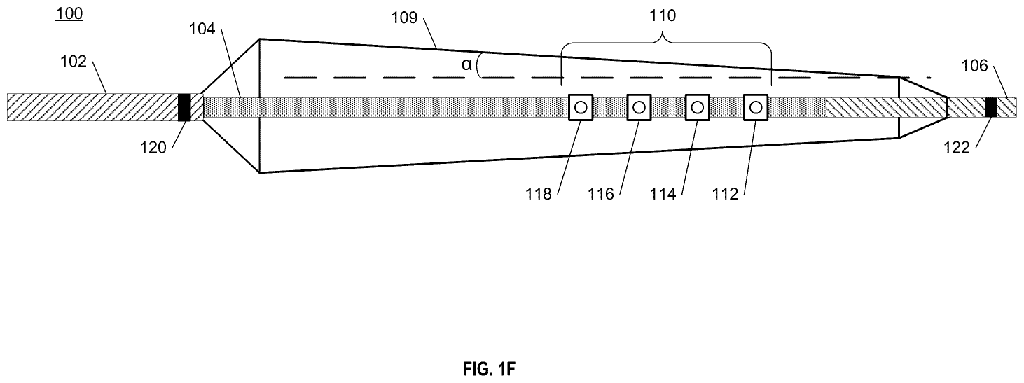

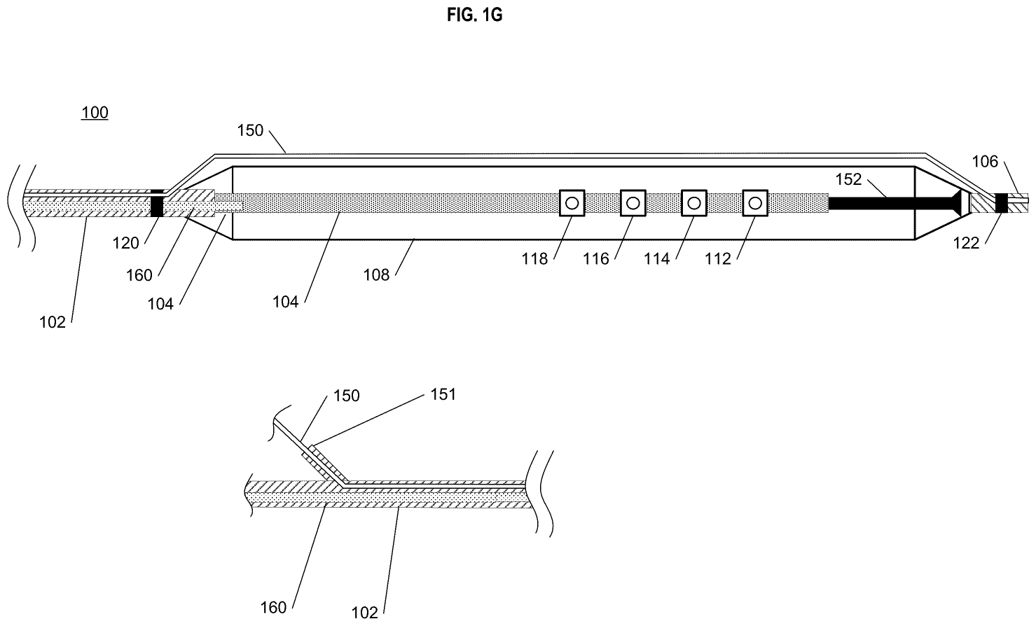

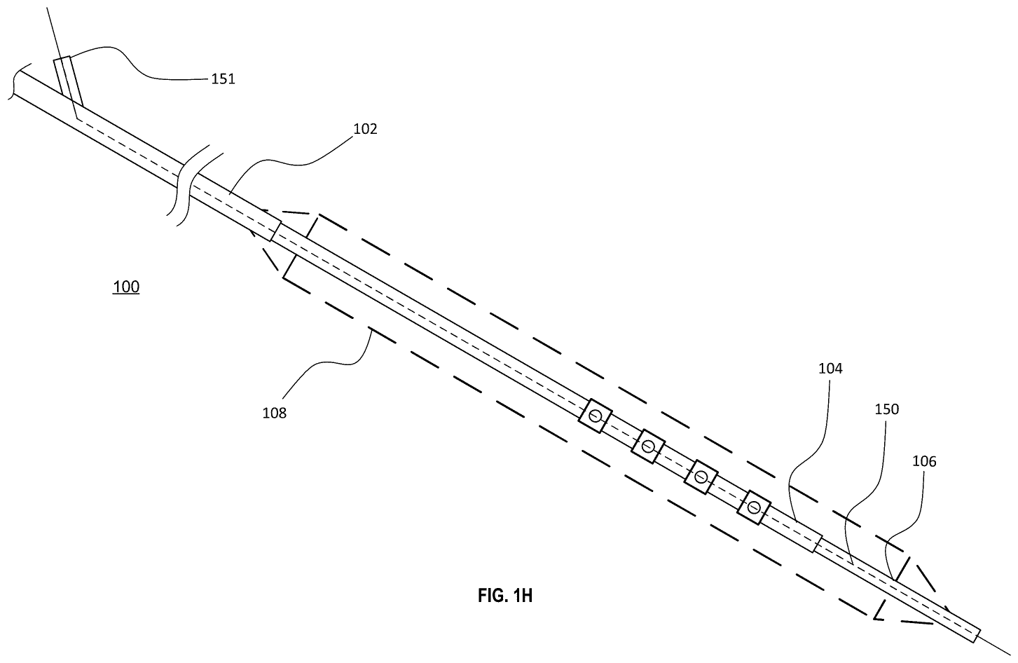

FIG. 1A illustrates an exemplary catheter 100 having a movable emitter array 110 inside an angioplasty balloon 108 at a proximal position, according to one or more embodiments. The catheter 100 includes an outer shaft (or outer elongate member) 102, a movable emitter carrier 104, and an inner shaft 106. The movable emitter carrier 104 may be tubular and define an internal lumen. Similarly, the outer shaft 102 may be tubular and define a lumen. The movable emitter carrier 104 may be concentric with the outer shaft 102. The movable emitter carrier 104 may also be concentric with the inner shaft 106. The angioplasty balloon 108 is secured (e.g. adhered) at a proximal end to outer shaft 102 and secured at a distal end to inner shaft 106. The balloon 108 is illustrated as inflated, where the volume of balloon 108 is inflated using a conductive fluid (e.g., saline), a contrast medium, or a combination thereof. In some embodiments, inner shaft 106 is a hollow tube, which can move around and along a guidewire (not shown), where the guidewire can pass through the inner shaft 106 and direct the catheter 100 to a target anatomy and lesion within the vascular of a patient. The movable emitter array 110 is shown in an exemplary embodiment having a first emitter 112, a second emitter 114, a third emitter 116, and a fourth emitter 118. In alternative embodiments, the movable emitter array 110 can have more or fewer emitters, for example, any number of emitters from one to six emitters. Given a sufficient power supply and corresponding circuitry, the movable emitter array 110 can have more than six emitters. In some embodiments, the angioplasty balloon 108 has a diameter of at least 1 millimeter (mm) and up to 25 mm. In some embodiments, the angioplasty balloon has a working length of at least 10 mm and up to 350 mm. In some embodiments, the catheter 100 has a working length of at least 100 cm and up to 350 cm. The working length of the movable emitter carrier within the volume of the balloon may be up to the full length of the balloon. In some embodiments, a distance from the most proximal emitter to the most distal emitter may be up to the full working length of the balloon.

In some embodiments as illustrated, each of the emitters include a cylindrical sheath (alternatively referred to as a ring or a band) mounted on and surrounding the movable emitter carrier 104. The cylindrical sheaths are formed of an electrically conductive material (e.g., a metal or an alloy) that accordingly forms a first electrode surface of an electrode pair. The sheath includes a cut-out region (e.g., a central circular hole, an arcuate cut-out on the edge of the sheath, etc.) that provides an unobstructed electrical path to a second electrically conductive material, specifically a conductive member (e.g., a copper wire, a flat coil, etc.) positioned underneath or within the movable emitter carrier 104, that thereby forms a second electrode surface of the electrode pair. In this configuration of emitter, the conductive portion of the sheath can be referred to as an outer electrode, and the wiring can be referred to as an inner electrode. Electric current delivered across the emitters can jump across the space between the two electrode surfaces, also referred to as a “spark gap,” and generate a shock wave as described above. Either of the electrode surfaces can be the anode or the cathode, depending on the polarity of the pulse delivered across the emitters. Spark gaps may be spaced so as to be circumferentially offset along the movable emitter carrier to provide a more uniform sonic output in the circumferential direction.

In many implementations, each emitter will have two electrode pairs, where current can travel from one electrode pair to the other electrode pair on the emitter by travelling across the electrically conductive material of the sheath. The electrode pairs on each emitter can be positioned to generate shock waves in directly opposite directions (i.e., arranged 180 degrees apart from each other around the catheter), or the electrode pairs can be positioned to generate shock waves in a convergent or biased direction (i.e., arranged at less than 180 degrees apart from each other around the catheter). In alternative implementations, an emitter can have three, four, five, or six electrode pairs arranged around the circumference of the emitter. The various emitters of the movable emitter array 110 can be electrically connected to each other in series or in parallel, on one or more electrical channels.

As part of the catheter 100, the balloon 108 has a proximal end and a distal end, where the proximal end of the balloon 108 is attached to the balloon shaft 102 and where the distal end of the balloon 108 is attached to the inner shaft 106. The balloon 108 is attached at both locations such that the inner volume of the balloon 108 is sealed, for example with an adhesive, with a clamp, by making a heat seal, by making a pressure seal, or a combination thereof. At the proximal end, balloon 108 is attached around the circumference of balloon shaft 102 such that the space between balloon shaft 102 and movable emitter carrier 104 can be used as a channel for fluid to enter and inflate balloon 108 (and also to correspondingly egress and deflate balloon 108). In some embodiments, another type of sealed enclosure (e.g., a cap or a flexible polymer tube) may be used instead of an angioplasty balloon.

The catheter 100 can be deployed to a target location within a patient with balloon 108 in a deflated configuration, where all of the movable emitter carrier 104 resides in a withdrawn position within outer shaft 102. A guidewire can be used to direct catheter 100 to a target tissue for treatment. The balloon 108 can then be inflated at a pressure appropriate for shock wave therapy (e.g., about 4 atm or a pressure sufficient such that the outer surface of the balloon 108 is in contact with and generally conforming to the shape of the surrounding tissue). In various embodiments, the fluid used to fill the volume of the balloon 108 can be saline or other conductive fluid, a contrast medium, or a mixture thereof. The fluid that fills the balloon 108 can be delivered to the interior of the balloon 108 through the channel formed by the space between the balloon shaft 102 and the movable emitter 104. In alternative embodiments, a separate fluid lumen can be used to provide fluid. In some embodiments, a balloon is fixedly located at the distal region of an inner shaft, and only a movable emitter lumen is translatable between the outside of the balloon volume to the inside.

The materials that can be used for components such as the outer shaft 102, movable emitter carrier 104, and inner shaft 106 can be extruded or molded polymers, or functional equivalents that can be safely used in a patient body. Such materials may include polyether block amide (e.g., Pebax), polytetrafluoroethylene (PTFE), nylon, or other polymers.

With balloon 108 inflated, the movable emitter carrier 104 can then be moved to various positions along the length of inner shaft 106 (remaining within the volume of balloon 108) where the emitters of the movable emitter array 110 can then be fired to generate shock waves at desired locations proximate to sections of the target tissue (e.g., a calcified blood vessel, aortic or mitral valve tissue, etc.).

As illustrated, movable emitter array 110 includes four individual emitters in order from the most distal emitter to the most proximal emitter as positioned along emitter carrier 104, first emitter 112, second emitter 114, third emitter 116, and fourth emitter 118. In various implementations, each emitter of movable emitter array 110 can have one or more electrode pairs. In various implementations, all of the emitters of the movable emitter array 110 can be wired on one electrical channel, such that current is provided to and across all emitters during each cycle of firing, which as illustrated in FIG. 1A would mean that first emitter 112, second emitter 114, third emitter 116, and fourth emitter 118 are all on the same single electrical channel. Using only one electrical channel helps to minimize the necessary physical wiring required, minimizing the width of the catheter 100, and thus contributes to minimizing the profile of the overall catheter 100. In alternative implementations, the emitters can be wired on two or more electrical channels, for example using the illustrations of FIG. 1A, with first emitter 112 and second emitter 114 being on one channel and with third emitter 116 and fourth emitter 118 on a second channel. Using two or more electrical channels allows for more operational flexibility in which emitters are fired, for example, firing only the more distal pair of emitters on a catheter to directionally bias the shock waves generated. Similarly, the operational flexibility of using two or more electrical channels can allow for extending the longevity of the catheter device, for example, continuing to fire the more proximal pair of emitters if the more distal pair of emitters have ceased functioning.

FIG. 1B illustrates an exemplary catheter 100 having a movable array of emitters 110 inside an angioplasty balloon 108 at a distal position. As illustrated, the movable emitter carrier 104 is extended in a distal direction along the catheter 100, covering more of the surface of the inner shaft 106, while remaining within the volume of balloon 108. In practical applications, a physician may choose to begin a regimen of shock wave therapy with the movable emitter array 110 in a distal position as shown in FIG. 1B, in a proximal position as shown in FIG. 1A, or in an intermediary position, as deemed appropriate for the treatment of a subject patient and the target tissue. Also shown are marker bands, specifically proximal marker band 120 and distal marker band 122, which are positioned next to the proximal and distal ends of balloon 108 and can be viewed during a procedure under radiography (e.g., x-ray fluoroscopy). In order to be viewed under radiography, the marker bands can be made from radiopaque materials such as iodine, barium, tantalum, bismuth, palladium, platinum, iridium, stainless steel, or alloys, oxides, sulfates, or other combinations thereof. The positioning of proximal marker band 120 and distal marker band 122 at the proximal end and distal end of the balloon 108, respectively, allows an operator to view the location of the balloon and catheter 100 within a patient.

FIG. 1C illustrates a cross-sectional view of the exemplary catheter 100 and one of the movable emitters (as illustrated, first emitter 112) inside an inflated angioplasty balloon 108 as shown in FIG. 1B. FIG. 1C describes in further detail how the wires can be electrically connected to the emitters on the movable emitter carrier 104. First wire 124 rests within one of grooves 128 formed in movable emitter carrier 104 and is aligned with a first spark gap 112a formed in emitter ring 112. Similarly, second wire 126 rests within a separate one of grooves 128 formed in movable emitter carrier 104 and is aligned with a second spark gap 112b formed in emitter ring 112c. Both first wire 124 and second wire 126 are insulated wires, excepting that at the location where first wire 124 is aligned with first spark gap 112a and where and second wire 126 is aligned with second spark gap 112b, the wires 124, 126 are exposed such that each constitutes an electrode surface that pairs with the respective opposing electrode surface of emitter ring 112c. As such, first emitter 112 has two electrode pairs: one located at the first spark gap 112a between first wire 124 and emitter ring 112c, and the other located at the second spark gap 112b between second wire 126 and emitter ring 112c. In alternative implementations, first wire 124 and second wire 126 can be embedded within the material of the movable emitter carrier 104, providing for a minimization of the cross-sectional profile of the overall catheter 100. In other alternative implementations, first wire 124 and second wire 126 can be sandwiched or pressed between the movable emitter carrier 104 and each of the emitters. First wire 124 and second wire 126 can be secured to movable emitter carrier 104 by being adhered to the surface of movable emitter carrier 104, wrapped with a coating onto the surface of movable emitter carrier 104 (e.g., shrink wrapped with a polymer material that surrounds the wires and the lumen), or a combination thereof.

In some embodiments, on the movable emitter carrier, each emitter can be from three to twenty millimeters distant from each other. The emitters can be electrically grouped (wired together) together on various channels of a circuit, where the emitters can be wired individually, as pairs, as triplets, or so on, and wired in series or in parallel. The power source supplying electricity to the emitters can supply power in the range of from about 1,000 V to about 15,000 V.

FIG. 1D illustrates an exemplary wiring configuration for the exemplary catheter and movable emitters inside an angioplasty balloon, according to one or more embodiments. The expanded view illustrates a first conductor 123 that extends from a power supply (not shown) to a most proximal emitter 118. The emitter 118 is electrically connected to a first return conductor 127 that extends proximally to the power supply. Current from a high voltage pulse travels from the power supply to the most proximal emitter 118 via first conductor 123 to generate a shock wave at the emitter 118 (as described above). Current then travels to the second most proximal emitter 116 via a second conductor 125 to generate a shock wave at the emitter 116. This continues until shock waves have been generated at each electrode pair electrically connected to the emitters 116, 118. Current then travels via return conductors (e.g., first and second return conductors 127, 129) back to the power supply. Although only one hole (signifying the location of an electrode pair where shock waves may be generated) is shown for each of the emitters in the figures, a plurality of electrically connected electrode pairs are possible at each emitter. In some embodiments, these conductors include insulated wires that have conducting regions either connected to an electrode or whose conducting region surface form an electrode. In some embodiments, the conductors or wires are arranged such that shock waves are successively generated from the most distal emitter to the most proximal emitter.

FIG. 1E illustrates an exemplary catheter 100 having a movable array of emitters inside a lumen directly proximate to an angioplasty balloon, in a pair of configurations with the balloon inflated and deflated, according to one or more embodiments. As shown, first emitter 112, second emitter 114, third emitter 116, and fourth emitter 118 are all in a position on movable emitter carrier 104 within the outer shaft 102 and proximal of the balloon 108. A distal end of the movable emitter carrier 104 may partially extend into the volume of the balloon 108 when inflated or when deflated, or the movable emitter carrier 104 may be fully withdrawn within the circumference of the outer shaft 102 before and/or after deployment.

An advantage of having the emitters positioned within the outer shaft 102 during deployment to a target tissue site is that the deflated balloon 108 is able to be folded or crimped down to have a diameter that is effectively the same as the outer surface of inner shaft 106 during delivery. This diameter can be from 5-8 French in gauge. In some embodiments, the deflated outer diameter is, at most, 8 French in gauge. Accordingly, the catheter 100 has a smaller cross-sectional profile for delivery to a vessel or lesion than if emitters were present and statically located in the balloon 108 on the inner shaft 106. While such emitters as used in IVL devices can add as little as 1 French or less to the diameter of the overall catheter during deployment, this difference in cross-sectional profile is meaningful to be able to both traverse certain vascular lesions and to fit within other vascular delivery systems. Further, keeping the emitters withdrawn inside the outer shaft 102 while in a deflated balloon 108 configuration reduces the risk that the material of the balloon may snag or tear on the emitters while being moved through the patient vasculature.

FIG. 1F illustrates an exemplary catheter having a movable array of emitters 110 inside a tapered angioplasty balloon 109 at a distal position. The use of a tapered angioplasty balloon 109 where the distal end is narrower than the proximal end can provide for improved access to vasculature that may be partially obstructed with plaque (e.g., a chronic total occlusion “CTO”), where the narrow end of the tapered angioplasty balloon 109 can function as a wedge to push into and through the plaque. In such situations where the tapered angioplasty balloon 109 is proximate to or in contact with a device-facing surface of vascular plaque, the movable emitter carrier 104 may be positioned in a distal location, and shock waves can be generated to break down the calcification of the surrounding lesion at the entry point of that vascular plaque. The relaxing or loosening of the vasculature at that location can then allow for the tapered angioplasty balloon 109 to be pushed further along the vessel and through the vascular plaque to ultimately allow for shock waves to be generated along the whole length of the calcified lesion. A tapered angioplasty balloon may also be implemented in body lumens that naturally taper. In some embodiments, a tapered angioplasty balloon has a tapered working region with a tapering angle (e.g., angle α) up to 20 degrees.

In the examples shown in FIGS. 1A-1F, the catheter may be configured to be advanced into the body lumen over a guide wire (not shown), generally referred to as an “over-the-wire” or “OTW” arrangement. In alternative implementations, as illustrated in FIGS. 1G and 1H, the catheter may be arranged as part of a “rapid exchange” or “Rx” configuration, where instruments are swapped in and out of a patient through a larger lumen during a procedure. FIG. 1G illustrates a catheter 100 according to one or more embodiments of the disclosure. The catheter 100 of FIG. 1G includes a path for a guidewire 150 for one rapid exchange configuration. The guidewire 150 may extend from a distal end of catheter 100 in a lumen of inner shaft 106, exit the inner shaft 106 at a distal side of a balloon 108, reenter the catheter 100 at a proximal side of the balloon 108, and extend proximally inside a lumen of outer shaft 102. Such a configuration may better facilitate movement of the emitter carrier 104 both in and out of the balloon 108 and to different locations within the balloon 108. The emitter carrier 104 moves back and forth on inner rail 152, which is static, and may extend proximally to a handle of the catheter. The inner rail 152 may be bonded to a distal tip of the balloon to keep the Rx guidewire stable during treatment. A rapid exchange guidewire exit 151 may be proximal of a distal balloon 108 and distal of a proximal end (not shown in FIG. 1G) of the catheter 100. The catheter 100 may include a fluid lumen 160 that extends through outer shaft 102 for delivering fluid to and moving fluid from the interior of balloon 108. It should be readily understood that such a fluid lumen can be used in OTW configurations, as well as Rx configurations of the catheter 100.

FIG. 1H illustrates a catheter 100 in another configuration of a rapid exchange system, according to one or more examples. The catheter 100 of FIG. 1H includes an inner shaft 106 having a lumen for a guidewire 150, which extends through a balloon 108. The movable emitter member 104 slidably moves along inner shaft 106 and may be retractable into outer shaft 102. The catheter 100 includes a guidewire exit 151 that is distal of a proximal catheter end (not shown in FIG. 1H). Advantageously, the guidewire 150 in both of the configurations shown in FIGS. 1G and 1H does not need to extend along the entire length of catheter 100.

FIG. 1I illustrates a cross-sectional view of an exemplary catheter 2000 at a region between a proximal handle and a movable emitter assembly, according to one or more embodiments. Catheter 2000 includes an inner shaft 2010. In one or more embodiments, the inner shaft 2010 may be tubular and define a guidewire lumen that accommodates a guidewire (not shown) over which catheter 2000 may be delivered. A movable emitter carrier 2020 is movably located over the inner shaft 2010. The movable emitter carrier 2020 may be tubular and define a lumen. The movable emitter carrier 2020 may include one or more wires (or another form of energy guides) that connect one or more emitters to a power source. The movable emitter carrier 2020 may be formed with grooves or lumens for wires (as described below) to reduce an overall profile of the catheter 2000. The movable emitter carrier 2020 is located between the inner shaft 2010 and an outer shaft 2030. In one or more embodiments, the outer shaft 2030 may be formed at least in part from a hypotube. The outer shaft 2030 also may include an outer layer 2040. Outer layer 2040 may include a braided structure and provide structural support to the catheter 2000 as it is navigated through a body lumen. In some embodiments, the movable emitter carrier 2020 has an inner diameter that is greater than an outer diameter of the inner shaft 2010. In some embodiments, the movable emitter carrier inner diameter is at least 50 micrometers (μm) greater than the outer diameter of the inner shaft 2010.

In various embodiments, a catheter having a movable emitter carrier having one or more shock wave emitters may include a relatively long enclosure (e.g., an angioplasty balloon). For example, the enclosure may have a working length that is 50%-200% longer than an end-to-end distance from a most proximal emitter to a most distal emitter. For example, the enclosure may have a working length l, and a distance between a most proximal shock wave emitter and a most distal shock wave emitter may be less than or equal to 0.5 l. To employ such a device, the balloon may be advanced through a body lumen to a lesion. A user may then inflate the balloon with a fluid (e.g., saline) to a relatively low pressure (e.g., less than 5 atm) or until the working region of the balloon apposes (i.e., contacts) the lesion. Upon inflation, an emitter assembly mounted to a movable emitter carrier may be advanced into the balloon volume to a first location (e.g., at a proximal location of the balloon) to treat the lesion or part of the lesion at the first location with shock wave therapy. The movable emitter carrier may then be farther advanced to a second location (e.g., at a more distal location than the first location) to treat a new lesion or a new region of the same lesion. In some embodiments, treatment may start at a distal region of the balloon or in a central region of the balloon. Advantageously, shock wave therapy may be customized for any particular lesion. For example, if there is a greater degree of calcification at a distal region of the balloon than at a proximal region of the balloon, more shock waves may be generated with the emitters at the more distal position than at the proximal position. By tuning the shock wave therapy in such a manner, unnecessary excess shock wave generation may be avoided, preserving the life of the device. Such devices also are less complicated in design than separately providing power (e.g., by electrical wires) to a large number of emitters located along a length of the angioplasty balloon.

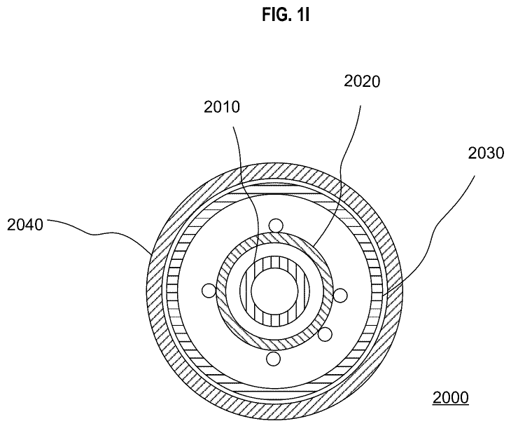

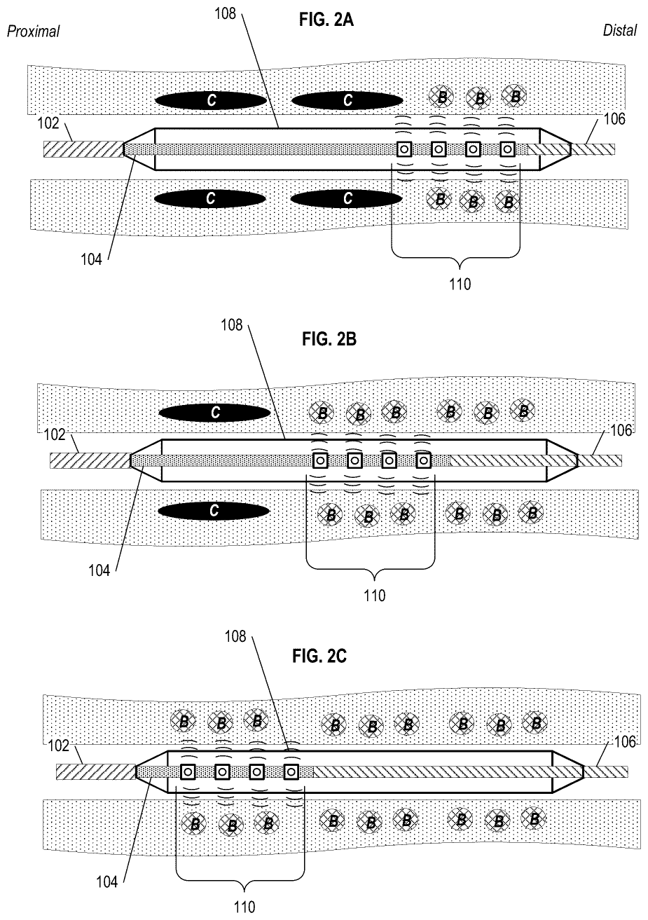

FIGS. 2A-2C illustrate an exemplary catheter within a vascular structure, the catheter having a movable array of emitters inside an angioplasty balloon shown at a distal position in FIG. 2A, at a central position in FIG. 2B, and at a proximal position in FIG. 2C. As illustrated, calcified regions of vascular tissue are indicated with a “C,” and regions of vascular tissue where the calcification has been subjected to shock waves and broken apart are indicated with a “B.” Starting with FIG. 2A, the inflated balloon 108 extends along the full length of the vascular tissue having calcification. The movable emitter carrier 104 extends along the length of inner shaft 106 to the distal end of balloon 108, with movable emitter array 110 positioned at the distal end of movable emitter carrier 104. Accordingly, the movable emitter array 110 is adjacent to the calcification at the distal end of the subject lesion in the vascular tissue. As illustrated, the emitters of movable emitter array 110 generate shock waves that break apart the calcification of the distal portion of the lesion.

Moving to FIG. 2B, the movable emitter carrier 104 has been pulled back in a proximal direction along the length of catheter 100 such that movable emitter array 110 is adjacent to calcification in the middle region of the subject lesion in the vascular tissue. As illustrated, the emitters of movable emitter array 110 generate shock waves that break apart the calcification in this middle portion of the lesion. It should be appreciated that the middle region of a long, calcified lesion may require repositioning of the movable emitter array 110 multiple times to deliver treatment to the complete length of middle regions of the calcified lesion.

Finally, moving to FIG. 2C, the movable emitter carrier 104 has been pulled back in a proximal direction along the length of catheter 100 such that movable emitter array 110 is adjacent to calcification at a proximal end of the subject lesion in the vascular tissue. As illustrated, the emitters of movable emitter array 110 generate shock waves that break apart the calcification of the proximal portion of the lesion.

It should be appreciated that more than one cycle of shock wave generation can be used to break down calcification in any given region of the calcified tissue, and that different regions of calcified tissue may need relatively more or less shock wave treatment as compared to each other. It can be further appreciated that the sequence of translating the movable emitter array 100 can proceed from distal-to-proximal, as described in FIGS. 2A-2C, but can alternatively proceed in a proximal-to-distal sequence, in a medial-to-proximal, or a medical-to-distal sequence. Further, the movable emitter array 110 can be moved to repeat treatment at locations along the calcified tissue two or more times.

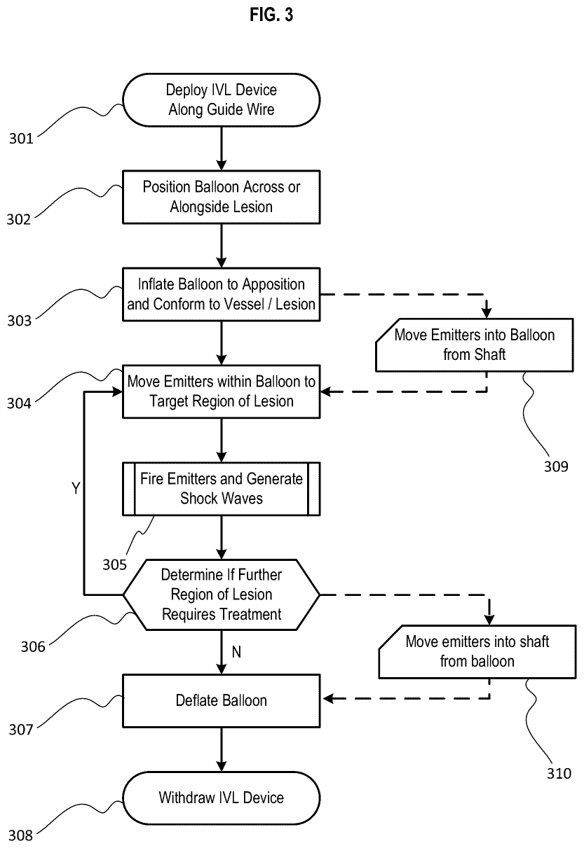

FIG. 3 is a flowchart describing a sequence of shock wave treatment of lesions using a movable array of emitters within an angioplasty balloon, according to one or more embodiments. At step 301, an IVL device is deployed along a guide wire into a body lumen (e.g., a blood vessel). At step 302, an enclosure (e.g., a balloon) of the IVL device is positioned across or alongside a lesion that is to be treated. At step 303, the enclosure is inflated until a working region of the balloon apposes and conforms to the body lumen wall and lesion. In some embodiments, the enclosure is inflated to a pressure less than 5 atm. In some embodiments, the enclosure is inflated to a pressure of at least 1 atm and at most 4 atm. At step 309, if the movable array of emitters is in a withdrawn position in an outer shaft (i.e., proximal of the balloon), the emitters are advanced into the enclosure to a first-target region of the lesion. At step 305, shock waves are generated at the movable array of emitters (e.g., by delivering a high voltage pulse or a laser pulse). At step 306, the user determines if a different region of the lesion requires shock wave treatment. If “yes,” then steps 304-306 are repeated; if “no,” then, at step 307, the enclosure is deflated. Upon deflation, the IVL device is withdrawn at step 308. Optionally, before withdrawal of the IVL device and after shock wave treatment, the enclosure is inflated to a pressure sufficient to radially expand the body lumens. In some embodiments, this second inflation pressure is higher than the pressure in step 303. In some embodiments, the second inflation pressure is greater than 5 atm. In some embodiments, the second inflation pressure is at least 4 atm and at most 12 atm. In some embodiments, at step 310, the movable array of emitters may be withdrawn into the outer shaft before the enclosure is deflated and the catheter withdrawn. In some embodiments, the method further includes a step of imaging the lesion (e.g., by x-ray fluoroscopy, intravascular ultrasound, and/or optical coherence tomography. The method may also include the step of, between rounds of shock wave treatment, deflating and inflating the balloon with a liquid to remove any accumulated gas bubbles inside of the balloon.

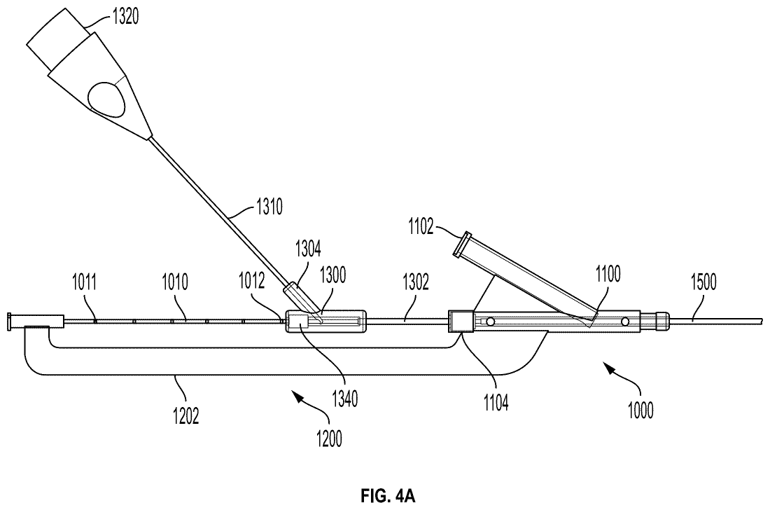

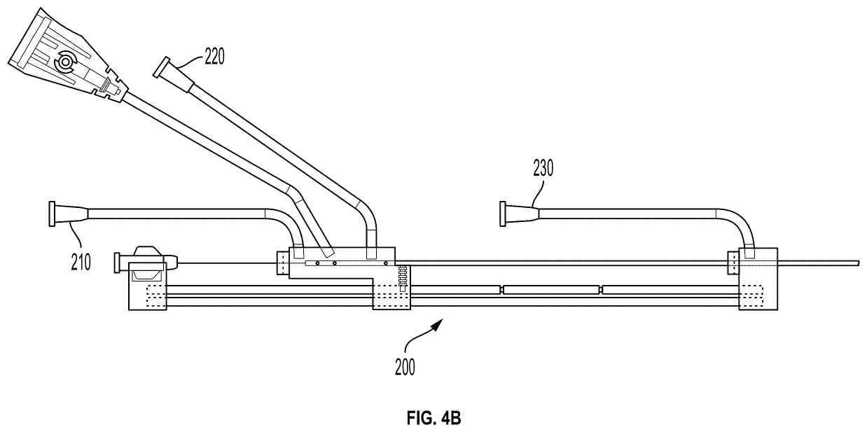

FIG. 4A illustrates an exemplary proximal handle 1000 for a catheter having a movable emitter assembly and balloon assembly, according to one or more embodiments. The handle 1000 may be used to advance or withdraw a movable emitter carrier 1302 that includes, at its distal region (not shown), the movable emitter assembly. The handle 1000 includes a fluid port 1102 that extends from a distal Y-arm 1100 and is fluidically connected to a lumen of the outer shaft 1500. The movable emitter carrier 1302 is slidably located inside of the outer shaft 1500 and extends proximally from a proximal end of the outer shaft 1500 through fluidically sealed connector 1104. In one or more embodiments, the connector 1104 may include a luer fitting.

The movable emitter carrier 1302 extends to a proximal Y-arm 1300 that is slidably located on an inner shaft 1010. The proximal Y-arm 1300 includes a power connector port 1304, through which a power source may be connected to the emitter assembly. For example, the emitter assembly may be electrically connected to a high voltage power supply by connector 1320 via conducting member 1310. In other implementations, one or more optical fibers may be connected to a light source (e.g., a laser) for supplying power for generating shock waves. In some embodiments, translation of the proximal Y-arm 1300 along the inner shaft 1010 by a distance d correlates with a longitudinal (in the distal-proximal direction) of the emitter assembly by the distance d.

Inner shaft 1010 extends to a proximal region of the handle 1000. In one or more embodiments, the handle 1000 includes a stabilizing structure 1200 that includes a bar 1202 that extends from a distal region of the handle 1000 to the proximal region. In some embodiments, the bar 1202 may be connected at its distal end to the distal Y-arm 1100. In one or more embodiments, the bar 1202 has a length that is approximately the same length as a working length of the balloon. The proximal Y-arm 1300 may be slidably translated along the inner shaft 1010 between the proximal region of the handle and the distal Y-arm 1100. When the proximal Y-arm 1300 is at a most proximal location of the inner shaft 1010, the emitter assembly may be entirely withdrawn in the outer shaft 1500. Distal translation of the proximal Y-arm 1300 moves the emitter assembly distally into the balloon towards a distal end of the balloon.

The proximal handle 1000 includes, in one or more embodiments, a connector 1340 in the proximal Y-arm. The connector 1340 may include a luer fitting. In one or more embodiments, the connector 1104 and the connector 1340 includes a silicone seal and/or a flexible valve that ensure a fluidic seal and help to maintain an internal pressure of the balloon. In some embodiments, the connector 1104 and the connector 1340 each include an O-ring to ensure a fluidic seal. These connectors also help to facilitate movement of the movable emitter carrier. Advantageously, silicone or silicone-type seals at these connectors may be self-sealing, and their use ensures a simple structure and mechanism to create a fluidic seal.

The proximal handle 1000 includes, in one or more embodiments, indicia located on the inner shaft 1010 or along the stabilizing bar 1202 to indicate to the user the position of the movable emitter assembly. For example, the proximal Y-arm 1300 being positioned at the proximal indicia 1011 may correlate to the emitter assembly being withdrawn from the balloon. The proximal Y-arm 1300 being positioned at the distal indicia 1012 may correlate to the emitter assembly being in a distal region of the balloon.

FIG. 4B illustrates another exemplary proximal handle 200 for a shock wave catheter, according to some embodiments of the disclosure. The handle 200 includes a first fluid port 210, a second fluid port 220, and a third fluid port 230, each fluidically connected by separate fluid lumens to a fillable enclosure. One or both of the first port 210 and second port 220 may be used to remove air and prime the catheter for use in shock wave therapy.

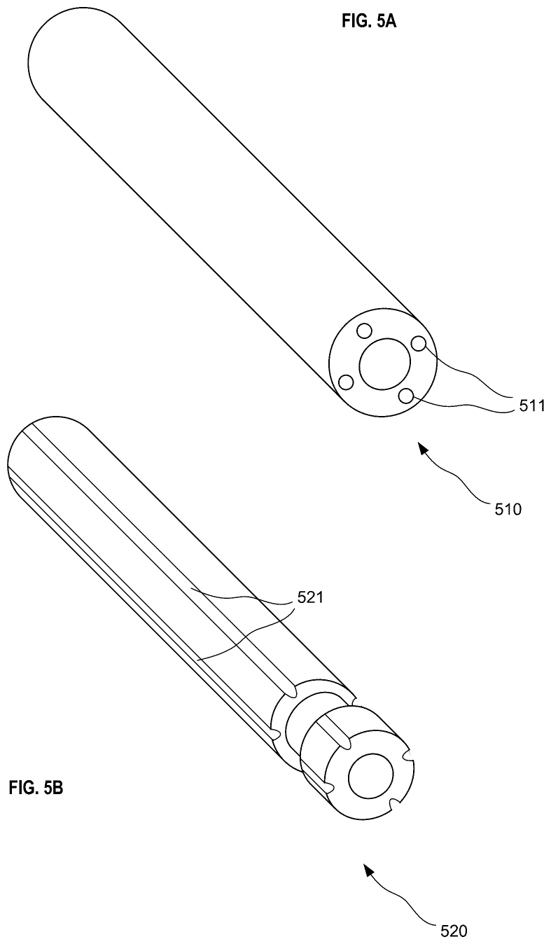

FIG. 5A illustrates an exemplary proximal section 510 of a movable emitter carrier, and FIG. 5B illustrates an exemplary distal section 520 of a movable emitter carrier, according to some embodiments. Proximal section 510 includes a plurality of conductor lumens through which conductors (e.g., wires) extend from a power supply. Having a cylindrical shaft with conductor lumens helps to provide the seals through the connectors at the proximal handle (e.g., luer fittings) described above. Distal section 520 includes a plurality of grooves that receive the same conductors. Grooves in the distal section 520 of the emitter shaft may help to provide a narrower profile at the distal end. In one or more embodiments, two dissimilar shafts are joined together (e.g., thermally joined) to form a movable emitter carrier having one or more conductor lumens in the proximal region and one or more conductor grooves in the distal region.

In one or more embodiments, an IVL catheter having movable emitters has a diameter of 8 French in gauge or less. In some embodiments, an IVL catheter having movable emitters has a diameter of 5 French in gauge or less.

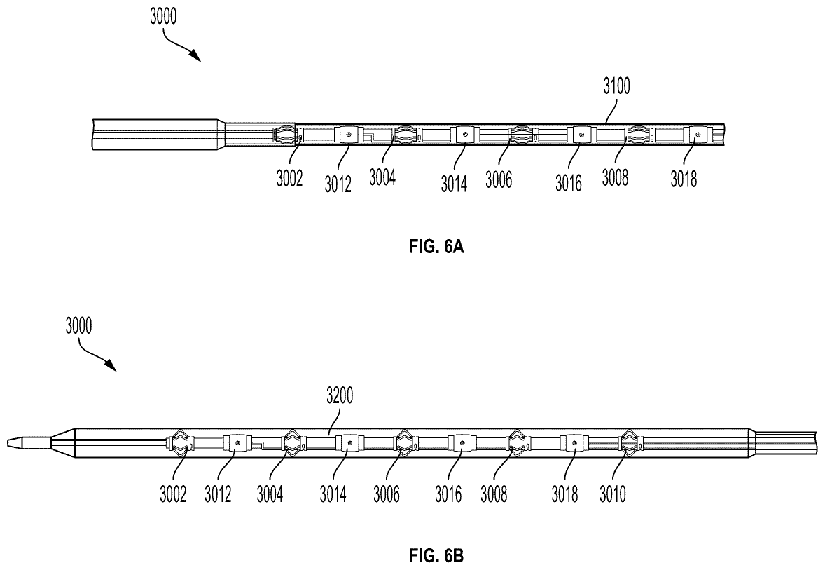

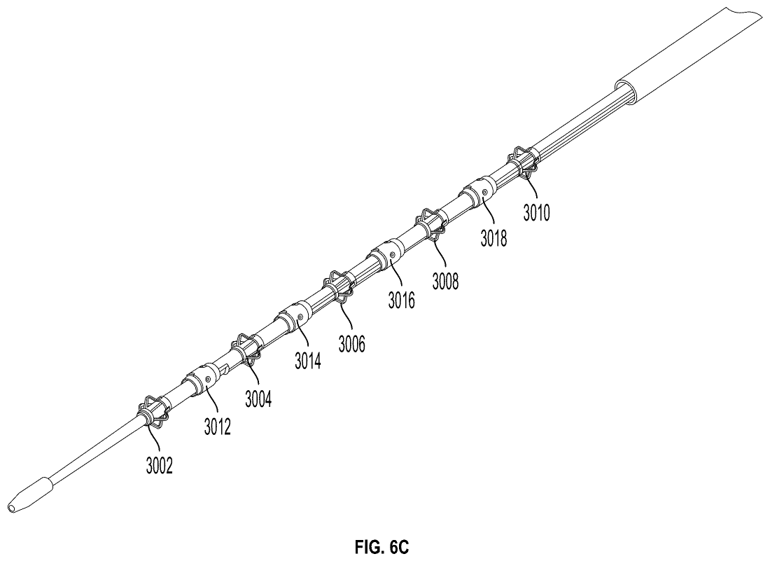

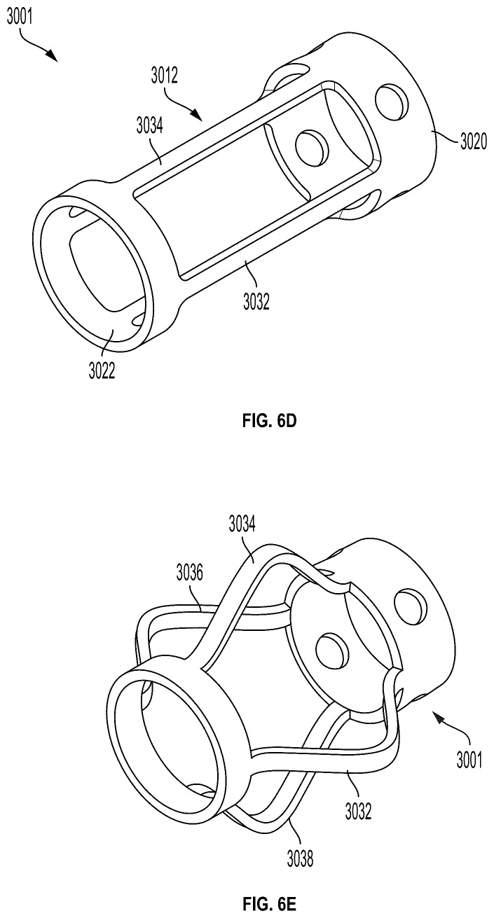

In some aspects of the invention, an IVL catheter includes an emitter centering feature including one or more shock wave emitter centering structures. FIGS. 6A-6C illustrate a catheter 3000 having centering structures 3002, 3004, 3006, 3008, 3010 adjacent to a shock wave emitter assembly including shock wave emitters 3012, 3014, 3016, 3018. FIG. 6A illustrates the centering structures in a radially compressed state. FIGS. 6B and 6C illustrate the centering structures in a radially expanded state, with FIG. 6C showing the catheter 3000 without an enclosure for clarity. The centering structures 3002, 3004, 3006, 3008, 3010 are in a radially compressed state when the emitter assembly of emitters 3012, 3014, 3016, 3018 is sheathed inside outer shaft 3100. The centering structures 3002, 3004, 3006, 3008, 3010 are in a radially expanded state when the emitter assembly is advanced out of outer shaft 3100 and into enclosure 3200. The centering structures 3002, 3004, 3006, 3008, 3010 myself-expand radially when unsheathed. When expanded, the centering structures 3002, 3004, 3006, 3008, 3010 contact an inner surface of the enclosure to space the emitters 3012, 3014, 3016, 3018 from the inner surface of enclosure 3200. In some embodiments, when radially expanded, the centering structures 3002, 3004, 3006, 3008, 3010 space each of the emitters 3012, 3014, 3016, 3018 from the inner surface of the enclosure 3200 by a distance of at least 0.1 mm. In some embodiments, the centering structures space one or more emitters by a distance of at least 0.5 mm from the inner surface of the enclosure. In some embodiments, the centering structures space one or more emitters by a distance of at least 2 mm from the inner surface of the enclosure. In some embodiments, a distance necessary for spacing of one or more shock wave emitters from an inner surface of an enclosure may depend on the enclosure's material properties (e.g., thermal degradation properties) and/or the energy output of the emitters.

In some embodiments, an IVL catheter includes a plurality of shock wave emitters and a plurality of emitter centering structures. The number of emitter centering structures may be one more than the number of shock wave emitters. An emitter centering structure may be located more proximally than a most proximal emitter. An emitter centering structure may be located more distally than a most distal emitter.

During delivery through a body lumen, the centering structures may be sheathed inside outer shaft 3100 in a radially compressed state. When enclosure 3200 is adjacent a lesion to be treated, the enclosure may be expanded. The emitters and centering structures may then be moved inside of the enclosure. Upon introduction into the wider diameter enclosure, the centering structures may self-expand until the centering structure contacts an inner surface of the enclosure. Centering the emitters, and consequently spacing the enclosure from the intense heat associated with shock wave generation, may be important to preserving the integrity of the enclosure (e.g., an angioplasty balloon).

FIGS. 6D and 6E illustrate an exemplary centering structure 3001 alone, according to one or more embodiment. FIG. 6D illustrates the centering structure 3001 in a radially compressed state. FIG. 6E illustrates the centering structure 3001 in a radially expanded state. The centering structure 3001 includes a first end 3020 and a second end 3022. Each of the ends may include a band (e.g., an annular band, a discontinuous band, or another type of band). The first end 3020 and second end 3022 may be connected by a self-expanding region 3030. The self-expanding region 3030 may include beams 3032, 3034, 3036, 3038. The beams 3032, 3034, 3036, 3038 may be flexible beams. The beams 3032, 3034, 3036, 3038 may be made of a material that can be radially compressed (i.e., straightened) and then be restored to the radially-expanded state. In some embodiments, the beams are made of nitinol, another nickel alloy, or another elastic material. The beams may have a width of 20-250 micrometers (μm). While centering structure 3001 is shown with four beams, other numbers of beams are possible, such as two, three, five, six, or more beams. When radially expanded, the centering structure may have a maximum diameter at least 0.5 mm larger than when radially compressed. In some examples, the maximum diameter of the centering structure in the radially expanded state is at least 0.75 mm larger than when radially compressed.

One or neither of the first end 3020 and second end 3022 may be fixedly adhered to a movable emitter shaft. By having at least one free end, the centering structure 3001 is free to be expanded in the enclosure or compressed when sheathed in an outer shaft.

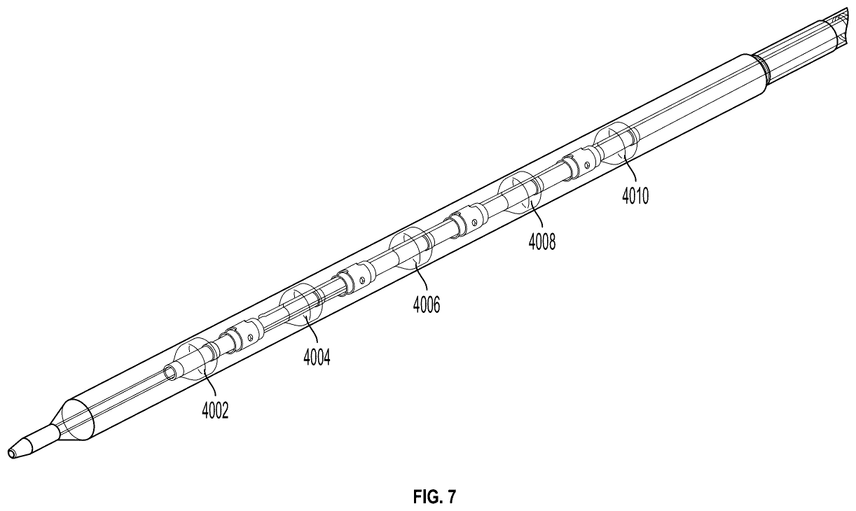

FIG. 7 illustrates an IVL catheter 4000 having an emitter centering feature including one or more centering structures 4002, 4004, 4006, 4008, 4010, according to one or more embodiments. Each of the centering structures 4002, 4004, 4006, 4008, 4010 includes an inflatable enclosure (e.g., a balloon) that is fluidically connected by a fluid lumen to a proximal fluid port. Similar to the centering structures shown in FIGS. 6A-6E, the centering structures 4002, 4004, 4006, 4008, 4010 function to space shock wave emitters from the enclosure. The inflatable enclosures of these centering structures may be made of a semi-compliant or non-compliant material.

In one or more embodiments, a centering feature may include one or more radially expandible porous (e.g., fibrous and/or polymeric) structures positioned adjacent and/or between shock wave emitters. Each porous structure may be configured to be in a radially expanded state and exert sufficient radial force when positioned inside of a relatively compliant enclosure (e.g., a semi-compliant or compliant angioplasty balloon) and to be in a radially collapsed state when positioned inside of a relatively non-compliant shaft or tubing.

It should be noted that the elements and features of the example catheters illustrated herein may be rearranged, recombined, and modified without departing from the present invention. For instance, the figures illustrate example electrode assemblies, the present disclosure is intended to include catheters having a variety of electrode configurations, and the number, placement, and spacing of the emitters and electrode pairs can be modified without departing from the subject invention.

Although the electrode assemblies and catheter devices described herein have been discussed primarily in the context of treating occlusions and lesions in coronary vasculature, the electrode assemblies and catheters herein can be used for a variety of occlusions and peripheral vasculature (e.g., above-the-knee, below-the-knee, iliac, carotid, etc.), other anatomy can be treated using IVL. For further examples, implementations of the embodiments disclosed herein may be used for treating soft tissues, such as cancer and tumors (i.e., non-thermal ablation methods), blood clots, fibroids, cysts, organs, scar and fibrotic tissue removal, multi-morphology tissue, or other tissue destruction and removal. Electrode assembly and catheter designs could also be used for neurostimulation treatments, targeted drug delivery, treatments of tumors in body lumens (e.g., tumors in blood vessels, the esophagus, intestines, stomach, or vagina), wound treatment, non-surgical removal and destruction of tissue, or used in place of thermal treatments or cauterization for venous insufficiency and fallopian ligation (i.e., for permanent female contraception).

In one or more examples, the electrode assemblies and catheters described herein could also be used for tissue engineering methods, for instance, for mechanical tissue decellularization to create a bioactive scaffold in which new cells (e.g., exogenous or endogenous cells) can replace the old cells; introducing porosity to a site to improve cellular retention, cellular infiltration/migration, and diffusion of nutrients and signaling molecules to promote angiogenesis, cellular proliferation, and tissue regeneration similar to cell replacement therapy. Such tissue engineering methods may be useful for treating ischemic heart disease, fibrotic liver, fibrotic bowel, and traumatic spinal cord injury (SCI). For instance, for the treatment of spinal cord injury, the devices and assemblies described herein could facilitate the removal of scarred spinal cord tissue, which acts like a barrier for neuronal reconnection, before the injection of an anti-inflammatory hydrogel loaded with lentivirus to genetically engineer the spinal cord neurons to regenerate.

As provided herein, it should be appreciated that any disclosure of a numerical range describing dimensions or measurements such as thicknesses, length, weight, time, frequency, temperature, voltage, current, angle, etc. is inclusive of any numerical increment or gradient within the ranges set forth relative to the given dimension or measurement.

Further, while the emitters disclosed in examples herein have a construction typically having two electrode pairs on each emitter, it is contemplated to also include emitters having three electrode pairs (for example, circumferentially separated from each other by 120 degrees), four electrode pairs (for example, circumferentially separated from each other by 90 degrees), five electrode pairs (for example, circumferentially separated from each other by 72 degrees) , six electrode pairs (for example, circumferentially separated from each other by 60 degrees), and so on. There may be physical limitations to the construction of such emitter assemblies relating to the size and arrangement of wiring, the ability to deliver sufficient power, the erosion profile of electrodes, and so on it is within the scope of the present disclosure that such emitters may be successfully developed with improvements in manufacturing capabilities.

Furthermore, numerical designators such as “first,” “second,” “third,” “fourth,” etc. are merely descriptive and do not necessarily indicate a relative order, location, or identity of elements or features described by the designators. For instance, a “first” shock wave may be immediately succeeded by a “third” shock wave, which is then succeeded by a “second” shock wave. As another example, a “third” emitter may be used to generate a “first” shock wave and vice versa. Accordingly, numerical designators of various elements and features are not intended to limit the disclosure and may be modified and interchanged without departing from the subject invention.

It will be understood that the foregoing is only illustrative of the principles of the invention, and that various modifications, alterations, and combinations can be made by those skilled in the art without departing from the scope and spirit of the invention. Any of the variations of the various catheters disclosed herein can include features described by any other catheters or combination of catheters herein. Furthermore, any of the methods can be used with any of the catheters disclosed. Accordingly, it is not intended that the invention be limited, except as by the appended claims.

1. A catheter for treating an occlusion in a body lumen, the catheter comprising:
an outer elongate member including a fluid lumen;
a flexible enclosure secured to a distal region of the outer elongate member, the flexible enclosure being fillable with a conductive fluid via the fluid lumen;
an inner elongate member, positioned within the outer elongate member and extending through the flexible enclosure to a distal region of the flexible enclosure; and
a movable emitter member having an emitter assembly mounted thereon and connected to a power source, the movable emitter member located between the inner elongate member and the outer elongate member and movable in a longitudinal direction between the inner elongate member and the outer elongate member.

2. The catheter of claim 1, wherein, in a first configuration of the movable emitter member, the emitter assembly is at a first location, and, in a second configuration of the movable emitter member, the emitter assembly is at a second location more distal of the first location.

3. The catheter of claim 2, wherein the emitter assembly is located proximal of the flexible enclosure in the first configuration, and the emitter assembly is located within the flexible enclosure in the second configuration.

4. The catheter of claim 2, wherein:
the flexible enclosure has a working length l,
the emitter assembly comprises a plurality of shock wave emitters, and
a distance between the most proximal shock wave emitter and the most distal shock wave emitter is less than or equal to 0.5 l.

5. The catheter of claim 1, further comprising a proximal hub, wherein the outer elongate member, the inner elongate member, and the movable emitter member extend to the proximal hub and translation of the movable emitter member along the inner elongate member at the proximal hub moves the emitter assembly.

6. The catheter of claim 5, wherein translation of the movable member by a distance d at the proximal hub moves the emitter assembly by the distance d in the longitudinal direction.

7. The catheter of claim 5, wherein the proximal hub comprises a distal end opening fluidically sealed to a proximal end of the outer elongate member.

8. The catheter of claim 5, wherein the inner elongate member includes indicia that correlate to the longitudinal position of the emitter assembly within the flexible enclosure.

9. The catheter of claim 5, wherein:
the proximal hub comprises a distal diaphragm seal and a proximal diaphragm seal,
the inner elongate member and the movable emitter member each extend through the distal diaphragm seal, and
the inner elongate member extends through the proximal diaphragm seal.

10. The catheter of claim 5, wherein the proximal hub comprises a position stabilizer with a first anchor at a first location of the proximal hub and a second anchor at a second location of the proximal hub, more proximal of the first location, and the movable emitter member includes a proximal end translatable between the first anchor and the second anchor.

11. The catheter of claim 1, wherein at least one of the inner elongate member and the movable emitter member comprises a polytetrafluoroethylene.

12. The catheter of claim 1, wherein the inner elongate member has an outer diameter d1 and the movable emitter member includes a lumen having a diameter d2 that is at least 0.002 inch greater than d1.

13. The catheter of claim 1, wherein the emitter assembly comprises an emitter centering member including:
a proximal band;
a distal band; and
a plurality of flexible beams connecting the proximal band to the distal band, each beam having a central region that extends radially outward.

14. The catheter of claim 13, wherein, the flexible enclosure includes an inflated state and a deflated state, and, when the flexible enclosure is in the inflated state, the central region extends radially farther outward than when the flexible enclosure is in the deflated state.

15. The catheter of claim 13, wherein the emitter assembly comprises one or more shock wave emitters and the central regions of the plurality of beams space the one or more shock wave emitters from a wall of the flexible enclosure.

16. The catheter of claim 13, wherein the emitter centering member comprises one or more of a polymer and a metal.

17. The catheter of claim 1, wherein the flexible enclosure comprises an angioplasty balloon having a working length l of at least 50 mm.

18. A method of performing intravascular lithotripsy, the method comprising:
introducing a catheter into a body lumen, the catheter including:
an elongate member extending from a catheter proximal end to a catheter distal end,
a movable emitter member movable along the elongate member and comprising a shock wave emitter assembly, and
an inflatable flexible enclosure secured to a distal region of the elongate member;
advancing the catheter through the body lumen until the flexible enclosure is adjacent an occlusion of the body lumen;
inflating the flexible enclosure with a fluid via a fluid lumen of the catheter;
moving the emitter member along the elongate member; and
supplying power to the shock wave emitter assembly to generate one or more shock waves.

19. The method of claim 18, wherein moving the emitter member includes moving the emitter member such that the shock wave emitter assembly moves between a first location proximal of the flexible enclosure and a second location within the flexible enclosure.

20. The method of claim 18, wherein moving the emitter member includes moving the emitter member such that the shock wave emitter assembly moves between a first location within the flexible enclosure and a second location within the flexible enclosure.

21. The method of claim 18, further comprising imaging the body lumen with at least one of x-ray fluoroscopy, optical coherence tomography, and intravascular ultrasound.

22. The method of claim 18, wherein moving the emitter member includes moving a proximal end of the emitter member.

23. The method of claim 18, further comprising correlating movement of the emitter assembly to movement of the emitter member at its proximal end by using indicia located along a proximal region of the elongate member.

24. An emitter centering member for an IVL catheter, the emitter centering member comprising:
a proximal band;
a distal band longitudinally spaced from the proximal band; and
a plurality of flexible beams connecting the proximal band to the distal band, wherein each flexible beam includes a center region that compressibly extends laterally outward.

25. A handle for an IVL device, the handle comprising:
a proximal end;
an elongate member extending from the proximal end;
a movable arm movably located on the elongate member and including a proximal diaphragm seal and a power port;
a distal arm located distal of the movable arm and including a distal diaphragm seal and a fluid port; and
a stabilizing bar that extends from the proximal end to the distal arm.

26. The handle of claim 25, wherein the movable arm includes a second fluid port.

27. The handle of claim 26, wherein the movable arm includes a third fluid port.

28. A catheter for treating an occlusion in a body lumen, the catheter comprising:
an elongate member including a fluid lumen;
a flexible enclosure secured to a distal region of the elongate member, the flexible enclosure being fillable with a conductive fluid via the fluid lumen and having a working length l; and
a movable emitter member having an emitter assembly mounted thereon and connected to a power source, the emitter assembly comprising a plurality of emitters,
wherein a distance from a most proximal emitter to a most distal emitter is less than or equal to l/2 and the movable emitter member is longitudinally translatable within the flexible enclosure.
